(19) United States
(12) Patent Application Publication
(10) Pub. No.: US 2005/0192341 A1
(43) Pub. Date: Sep. 1, 2005

(54) Non-psychotropic cannabinoids for prevention of cognitive impairment

(76) Inventors: Seth Kindler, Tel Aviv (IL); Aaron Garzon, Rehovot (IL); George Fink, Tel Aviv (IL)

Correspondence Address: WINSTON & STRAWN LLP, 1700 K STREET, N.W., WASHINGTON, DC, 20006, US

(21) Appl. No.: 11/073,250

(22) Filed: Mar. 4, 2005

Related U.S. Application Data
(63) Continuation of application No. PCT/IL03/00735, filed on Sep. 4, 2003.
(60) Provisional application No. 60/408,958, filed on Sep. 5, 2002.

(51) Int. Cl. A61K 31/353

(52) U.S. Cl. 514/454

(57) ABSTRACT

The present invention relates to method of using pharmaceutical compositions that contain, as an active ingredient, non-psychotropic cannabinoids for preventing, alleviating or diminishing cognitive impairment resulting from certain types of surgery, diseases or viral infections, fetal distress, pre-term or low weight delivery, from medical intervention such as certain types of medication, irradiation or electroconvulsive therapy, or for prophylactic use in populations exhibiting mild cognitive impairment, and other populations at risk for chronic neurodegenerative diseases.

### CROSS-REFERENCE TO RELATED APPLICATIONS

This application is a continuation of International application PCT/IL2003/000735 filed Sep. 4, 2003, and claims the benefit of U.S. Provisional application 60/408,958 filed Sep. 3, 2002. The entire content of each document is expressly incorporated herein by reference thereto.

### FIELD OF THE INVENTION

The present invention relates to pharmaceutical compositions comprising as an active ingredient non-psychotropic cannabinoids for preventing, alleviating or diminishing cognitive impairment resulting from certain types of surgery, diseases or viral infections, abnormal peri-natal conditions, medical interventions such as certain types of medications, and for prophylactic use in populations exhibiting mild cognitive impairment, and other populations at risk for chronic neurodegenerative diseases.

### BACKGROUND OF THE INVENTION

Mild cognitive impairment (MCI) is a recognized disorder and has recently become a focus for research and clinical attention. According to current neurological nomenclature MCI is defined by the following criteria: subjects with memory impairment beyond that expected for age and education, with normal general cognitive and functional activities, and yet not demented (Peterson R. C. et al. , Arch. Neurol. 56: 303,1999).

The mild impairment involves memory and generally other cognitive domains that are more impaired in dementia. In addition the common activities of daily living (ADL) are intact, but there may be subtle impairment in very complex ADL. MCI is further categorized as two subtypes, MCI-amnestic and MCI-other, depending on the presence or absence of amnesic components. Numerous studies have demonstrated that the presence of MCI, in particular the amnestic subtype, may be a risk state for the development of dementia. The fact that up to 15% of MCI patients, generally the more impaired, will develop Alzheimer's disease (AD), as opposed to 1% in the corresponding healthy population, prompted clinicians to believe that treatment of MCI may prevent, delay or even reverse disease-associated brain deterioration.

Other neurodegenerative disorders, such as Lewy-Body dementia (LWD), Parkinson's disease (PD) and vascular dementia (VaD), progress from undetectable cognitive impairment to insidious cognitive and behavioral decline, culminating in the development of severe dementia. It is believed that being in a transition state the prodromal population should be targeted for early intervention in order to prevent the cognitive impairment and its progression toward dementia. The currently envisioned symptomatic therapeutic approaches include cholinesterase inhibitors, glutamatergics, NMDA antagonists, AMPA modulators, hormone replacement, nootropics, anti-inflammatory drugs, antioxidants and BETA-AMYLOID cascade inhibitors. The research is ongoing and the results of clinical trials remain controversial.

MCI is also associated with several non-dementia related conditions including cardiovascular diseases, haemodynamic shock, atherosclerosis, ischemic events, traumatic brain injury (TBI), demyelinating disorders, Huntington's disease (HD), Amyotrophic Lateral Sclerosis (ALS), cancer, autoimmune diseases, multiple sclerosis (MS), mood disorders, epilepsy and chronic infections.

Viral infections, for instance, are often associated with cognitive impairment (CI), human immunodeficiency virus (HIV), herpes simplex virus (HSV), hepatitis C virus (HCV) and cytomegalovirus (CMV) being the most often reported infective agents that correlate with cognitive impairment. At least 20% of patients newly diagnosed for CMV suffer from CI, and this figure only rise with disease progression. An estimated 30% of persons diagnosed with HIV develop cognitive changes and an additional 15-20% develop more profound debilitating dementia. The worldwide prevalence of these viral infections is significant with about 170 million people for HCV, over 50 million people infected with HIV, 15-50% for HSV and over 50% for CMV. Even with a conservative estimate of 20% of virally infected person who will develop CI, virally induced CI represent an important challenge for the medical community.

Other medical conditions are associated with an increased risk of CI. For example, children born pre-term or low weight or having undergone fetal distress constitute an additional population at risk of developing CI. The probability of any of these abnormal peri-natal conditions is increased in case of birth to women over thirty-five or in multiple gestation, whose prevalence is rising with the diffusion of assisted reproductive technology. In the United States alone, the rate of pre-term delivery, i. E. the birth of an infant before 37 completed weeks of gestation, and low birth weight, i. e. body weight inferior to 2500 grams, is about 12% of live births and this high proportion represents a public health concern.

Finally, sometime the treatment, and not the disease, is responsible for the resulting cognitive impairment. Therapy induced CI fall into two categories: procedure or drug induced. It is estimated that medications contribute up to 40% of all cases of CI. The medication induced cognitive impairment is predominantly observed in the elderly, but is not restricted to this population. More than 50% of hospitalized elderly patients will develop medication-induced CI. The medicaments most often cited in the context of drug-induced cognitive impairment are the anesthetic agents, the cytokines used in immunotherapy, the drugs used in chemotherapy, the anti-cholinergic medications, narcotics, opioids, tricyclic antidepressants, anticonvulsants, anti-epileptic drugs, histamine H2 receptor antagonists, cardiac medications, corticosteroids, non steroidal anti-inflammatory drugs (NSAIDs), anti-viral agents, anti-parasitic agents and antibiotics. Medication-induced CI is generally but not always considered reversible, the change in mental status coinciding with the period of drug use and improvement being observed upon cessation of treatment or shortly thereafter. The strategies used to circumvent this condition include dose reduction and change of medication, but these approaches are not always applicable or effective, especially when chronic treatment is needed.

Procedure induced CI is observed for instance during surgical interventions, electroconvulsive therapy (ECT) and irradiation therapy. Over ten million surgical procedures are performed in the United States alone in adults over 60 years. In this population at risk the incidence of CI is between 30 to 50% for most types of surgery, with a clear correlation with increased age. ECT is a medical procedure in which a brief electrical stimulus is used to induce a cerebral seizure under controlled condition. More than 200,000 such procedures are performed each year in the United States. The degree of cognitive impairment is influenced by the electrode placement and by the type of current used. But even after optimization of these parameters most patients, 50-80%, receiving ECT develop CI, which will at least continue for the duration of the treatment over several weeks. In rare cases, CI may last for a considerably longer period from months to years. The prevalence of cancer is rising with estimates of about 20% of the population affected, about half receive radiation therapy, and 30-50% of them develop radiation-induced CI. These three examples emphasize the importance of the therapeutical procedure as an inducer of cognitive impairment.

The better-known example of procedure induced CI is during cardiac surgery with very high prevalence of post-operative cognitive impairment. Mortality after cardiac surgery (CS) has fallen steadily over recent years, however concern remains about the effect of this surgery on the brain. The neurological side effects were originally attributed to the use of cardiopulmonary bypass (CPB) during the procedure and were potentially caused by the release of atheromatous debris, small clots, lipid particles, particulate or gaseous microemboli during the surgery. Additional factors that play a role in the development of brain damage include: disturbed perfusion, metabolic derangement, and inflammatory responses. The number of microemboli has been linked to the likelihood of neuropsychological deterioration after surgery. Risk factors for cerebral changes after CS include age, gender, neurological disease, diabetes, and calcification of the aorta. These risk factors are important because patients undergoing CS are now older and tend to have a greater number of other morbid conditions. Changes in surgical technique, such as the introduction of arterial-line filters and membrane oxygenators, have led to a reduction of both microemboli and neuropsychological disturbance. Nevertheless, a recent study suggests that patients undergoing surgery even without cardiopulmonary bypass (off-pump procedure) are at no significantly reduced risk to develop cognitive impairment (Van Dijk D. et al., JAMA 287: 1405, 2002). Thus, the problem of post-operative cognitive impairment persists both for on-pump and off-pump procedures, prompting further studies on surgical technique and neuroprotective strategies.

Frank neurological impairment occurs in only about 6% of patients. This prevalence further increases when patients are undergoing combined surgical procedures. However, post-operative decline in cognitive performance is far more prevalent, and as many as 90% of the patients on cardiopulmonary bypass suffer from at least a transient deterioration.

Moreover, CI can be the undesirable result of a variety of surgical interventions, not only on the cardiovascular system. This is especially relevant in the elderly population, which is more predisposed to develop cognitive dysfunction after any major operation. During the last several years there has been growing recognition and awareness of the need for a prophylactic neuroprotective agent in patients undergoing procedures such as cardiac surgery including valvular surgery, carotid surgery, endarterectomy, endovascular therapy, aneurysm repair, orthopedic or prosthetic surgery, which have an increased risk of developing CI due to the cerebral ischemic events that occur during surgery.

Heightened awareness of the prevalence and seriousness of this complication has fostered a change in current surgical practice to minimize embolic injury. However despite these preventative methods the incidence of post surgical cognitive impairment remains high and mandates investigation of new treatments for the prevention of cerebral injury in very high-risk population.

The severity of cognitive defects is dependent not only on the primary ischemia and hypoxia caused by microemboli, but also on secondary brain cell death. Microembolization has been shown to induce a neuroinflammatory cascade triggered by the release of excitatory amino acid transmitters in particular glutamate. The glutamate-induced cell death cascade involves activation of glutamate receptors, of which the NMDA N-methyl-D-aspartate) subtype plays a pivotal role. The massive influx of calcium ions into cells, and the generation of free radicals, nitric oxide (NO), cytokines including tumour necrosis factor-alpha (TNF-α), and prostaglandins. These secondary insults determine the total amount of brain injury that occurs in addition to the primary brain injury. The extent of the primary brain injury cannot be controlled pharmacologically, and so attention has focused on moderating the extent of secondary brain injury, thus minimizing the extent of the final damage. The secondary damage that occurs as a result of an inflammatory reaction in the surrounding brain tissue may progress for days after the initial insult. The inflammation involves injured, yet potentially viable, brain cells that might ultimately die increasing the severity of cognitive impairment.

Though the primary neurological damage leading to neurological disorders may differ from one condition to another, the secondary damage might share underlying similarity with the causes of post-operative CI. Progress in genetics, epidemiology and early diagnosis of asymptomatic patients allows the identification of population at increased risk to develop such neurodegenerative disorders, such as AD. This population is an important target for prophylactic treatment since as many as 10% of the population in Western countries may develop such diseases.

Currently, no drug exists for preventing post-surgical cognitive impairment. Such compounds will be of therapeutical interest not only for the prevention, reduction or treatment of post-surgical cognitive impairment but also for the prophylactic treatment of populations exhibiting mild cognitive impairment, at risk for chronic neurodegenerative diseases. Thus, the present invention provides solutions to the long-felt unmet medical need for therapeutic means of intervening in or preventing cognitive impairment and its sequellae.

### SUMMARY OF THE INVENTION

The present invention relates to pharmaceutical compositions for prevention of cognitive impairment comprising as an active ingredient a non-psychotropic cannabinoid or derivative thereof that is characterized in that it possesses both neuroprotective and anti-inflammatory activity. The present invention relates to novel uses of any synthetic or natural cannabinoid which is essentially devoid of appreciable psychomimetic activity that acts both as a neuroprotective and an anti-inflammatory agent. Preferred compounds are cannabinoid analogs of the Δ⁶-tetrahydrocannabinol (THC) type having the (3S,4S) configuration. Currently preferred are synthetic non-psychotropic derivatives of dexanabinol, also known as HU-211, and dexanabinol itself.

Compounds having the general structure of tetrahydrocannabinoids having the 3S,4S configuration are disclosed in international application WO 01/98289. This family of compounds is based on derivatives of 1,1-dimethyl-(3S,4S)-7-hydroxy-Δ⁶-tetrahydrocannabinol, disclosed in U.S. Pat. No. 4,876,276 and denoted therein HU-211. HU-211 was subsequently assigned the trivial chemical name dexanabinol. These compounds are derivatives and analogues of essentially pure stereospecific (+) enantiomers, having the (3S,4S) configuration, of Δ⁶-tetrahydrocannabinol (THC) type compounds, devoid of any undesirable cannabimimetic psychotropic side-effects. These known compounds have been described as having neuroprotective and/or anti-inflammatory properties, among other beneficial activities as disclosed in U.S. Pat. Nos. 5,284,867, 5,521,215, 5,538,993, 5,635,530, 5,932,610, 6,096,740, 6,331,560, 6,545,041 and 6,610,737.

The inventors have now found that said known compounds are also effective in prophylactic administration to prevent or ameliorate conditions of post-operative cognitive impairment, disease induced, virally induced, therapy induced, neonatal cognitive impairment and onset of neurodegenerative diseases.

The compounds serving as active ingredient in the compositions of the present invention may operate via diverse mechanisms to provide the combined neuroprotective and anti-inflammatory properties.

The pharmaceutical compositions of the present invention will be useful in the prevention of post-operative, disease induced, virally induced, therapy induced, neonatal cognitive impairment.

The pharmaceutical compositions of the present invention will be useful in prophylactic treatment of populations at risk for neurodegenerative disorders, including patients suffering from mild cognitive impairment.

Accordingly, the present invention provides pharmaceutical compositions for preventing, alleviating or diminishing post-operative, disease induced, virally induced, therapy induced, and neonatal cognitive impairment comprising as an active ingredient a compound of general formula (1): 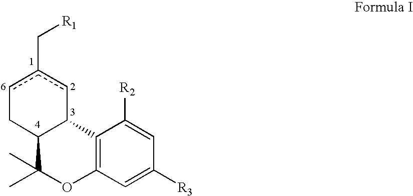
having the (3S,4S) configuration and being essentially free of the (3R,4R) enantiomer, wherein the dashed line indicates an optional C1-C2 or C6-C1 double bond, and wherein:
R₁ is selected from the group consisting of
   a) R′ where R′ is selected from the group consisting of
      A) a linear or branched, saturated or unsaturated, carbon side chain comprising 1-8 carbon atoms optionally interrupted by 1-3 heteroatoms, and
      B) a saturated or unsaturated cyclic moiety, an aromatic moiety or a heterocyclic moiety; the cyclic moiety having from 3-20 atoms comprising one or two-ringed structures, wherein each ring comprises 3-8 carbons, optionally interrupted by 1-4 heteroatoms, and optionally further substituted with one or more groups selected from
         i) a linear, branched or cyclic, saturated or unsaturated C₁-C₆ alkyl,
         ii) a linear, branched or cyclic, saturated or unsaturated C₁-C₆ alkoxy,
         iii) a linear, branched or cyclic, saturated or unsaturated C₁-C₆ alkylthio,
         iv) a halogen,
         v) carboxyl,
         vi) —CO₂—C₁-C₆ alkyl, wherein the alkyl can be linear, branched or cyclic, saturated or unsaturated,
         vii) keto,
         viii) nitro,
         ix) a saturated or unsaturated cyclic moiety, or an aromatic or a heterocyclic moiety comprising one or two ringed structures, wherein each ring comprises 3-8 carbons optionally interrupted by 1-4 heteroatoms, said heteroatoms each independently selected from the group consisting of N, O, and S; wherein each ring optionally is further substituted with one or more groups selected from i)-viii) as defined above,
   b) an amine or an amide substituted with at least one substituent as defined in R′ above,
   c) a thiol, a sulfide, a sulfoxide, a sulfone, a thioester or a thioamide optionally substituted with one substituent as defined in R′ above, and
   d) a hydroxyl or an ether —OR′ wherein R′ is as defined above;
R₂ is selected from the group consisting of
   a) a halogen,
   b) a linear, branched or cyclic, saturated or unsaturated C₁-C₆ alky, and
   c) —OR wherein R is selected from the group consisting of
      A) —R″, wherein R″ is hydrogen or a linear, branched or cyclic, saturated or unsaturated C₁-C₆ alkyl optionally containing a terminal —OR′″ or —OC(O)R′″ moiety wherein R′″ is hydrogen or a linear, branched or cyclic, saturated or unsaturated C₁-C₆ alkyl, and
      B) —C(O)R′″ wherein R′″ is as previously defined; and
R₃ is selected from the group consisting of
   a) a linear, branched or cyclic, saturated or unsaturated C₁-C₁₂ allyl,
   b) —OR′, in which R^{a} is a linear, branched or cyclic, saturated or unsaturated C₂-C₉ alkyl which may be substituted at the terminal carbon atom by a phenyl group, and
   c) a linear, branched or cyclic, saturated or unsaturated C₁-C₇ alkyl-OR′″ wherein R′″ is as previously defined;
and pharmaceutically acceptable salts, esters or solvates thereof.

The present invention further provides pharmaceutical compositions for preventing, reducing or delaying the onset of neurodegenerative disorders in populations at risk exhibiting mild cognitive impairment comprising as an active ingredient a compound of general formula I as previously defined.

The pharmaceutical compositions may contain in addition to the active ingredient conventional pharmaceutically acceptable carriers, diluents and excipients necessary to produce a physiologically acceptable and stable formulation.

The pharmaceutical compositions can be administered by any conventional and appropriate route including oral, parenteral, intravenous, intramuscular, subcutaneous, transdermal, intrathecal, rectal or intranasal.

Prior to their use as medicaments for preventing, alleviating or treating an individual in need thereof, the pharmaceutical compositions may be formulated in unit dosage forms. The selected dosage of active ingredient depends upon the desired therapeutic effect, the route of administration and the duration of treatment desired.

A further aspect of the present invention provides a method of preventing, alleviating or diminishing post-operative, disease induced, virally induced, therapy induced, neonatal cognitive impairment, by administering to a patient in need thereof a prophylactically and/or therapeutically effective amount of a pharmaceutical composition containing as an active ingredient a compound of general formula I as previously defined.

A further aspect of the present invention provides a method for preventing, reducing or delaying the deterioration of mild cognitive impairment to chronic neurodegenerative disorders, by administering to a patient in need thereof a prophylactically and/or therapeutically effective amount of a pharmaceutical composition containing as an active ingredient a compound of general formula I as previously defined.

A further aspect of the present invention relates to the use for the manufacture of a medicament for preventing, alleviating or diminishing post-operative, disease induced, virally induced, therapy induced, neonatal cognitive impairment, of a compound of general formula I as previously defined.

A further aspect of the present invention relates to the use for the manufacture of a medicament for preventing, reducing or delaying the deterioration of mild cognitive impairment to chronic neurodegenerative disorders, of a compound of general formula I as previously defined.

### BRIEF DESCRIPTION OF THE FIGURE

To assist in the understanding of the invention, and in particular of the data that are given in the examples, the following drawing figure is presented herein:

FIG. 1 shows the effect of dexanabinol administered prior to surgery on learning capacity (panel A) and on memory panel B) in animals subjected to four-vessel occlusion as measured using the Morris water maze model.

### DETAILED DESCRIPTION OF THE INVENTION

The present invention provides pharmaceutical compositions effective in prophylactic treatment of patients who suffer from medical conditions that may result in cognitive impairment, including but not limited to surgical operations, certain diseases or viral infections, certain therapeutic procedures or medications, abnormal peri-natal conditions and of patients at risk for the development of neurodegenerative disorders. The advantageous mechanisms of action are now disclosed to be the combined neuroprotective and anti-inflammatory activities of the therapeutic agents.

Increasing knowledge about the pathophysiology of ischemic or hypoxic brain injury gained from research and clinical trials has demonstrated that pretreatment, prior to the ischemic insult improves the efficacy of neuroprotective agents. Numerous studies have shown lengthening the period between insult and neuroprotective administration diminishes treatment benefits. From these findings it may be postulated that the ideal human settings in which to achieve the maximum benefit from a neuroprotective agent are those in which a human subject is at risk of cerebral ischemic or hypoxic injury expected to occur at a predictable time. During certain types of surgery, especially cardiac surgery (CS) with or without cardiopulmonary bypass (CPB), patients are at risk of multiple cerebral emboli during a limited, pre-determined period of time and frequently develop ischemia-related measurable cognitive impairment.

Additional medical conditions, beside surgery, have been shown to be associated with increased risk of cognitive impairment. In this case the CI is induced either by certain types of diseases, disorders or viral infections, or by certain types of treatments either by a medical procedure or by a medication administered. The secondary neurological injury leading to cognitive impairment is probably due to mechanisms distinct from cerebral ischemia.

The present invention relates to use of THC-type compounds which are characterized by an absolute stereochemistry at the positions 3 and 4 of the molecule (3S,4S), which is opposite to the (3R,4R) configuration in the natural series. The natural compounds of the (3R,4R) configuration produce undesirable psychotropic “cannabis” type effects, which preclude their use for other therapeutically interesting effects. The compounds of the invention being of the (3S,4S) configuration are substantially devoid of the undesired psychotropic effect and thus can be used for the treatment of various diseases and disorders. Thus, in the present specification and claims which follow the term “essentially free” qualitatively refer to (3S,4S) compounds of high optical purity substantially devoid of the undesired psychotropic effect lying with the (3R,4R) enantiomer. The quantitative criterion of the minimum acceptable degree of optical purity of an intended therapeutic enantiomer is dictated by the pharmacological potency of the opposite enantiomer. The higher the psychotropic activity of the opposite enantiomer, the stricter the requirement for optical purity. The enantiomeric pair HU-210 and HU-211, of (3R,4R) and (3S,4S) configuration, respectively, is an extreme example of such a situation, HU-210 being a synthetic cannabinoid at least one hundred times more psychoactive than natural Δ⁹-THC, the major active constituent in marijuana (Mechoulam R. et al., Tetrahedron Asymmetry 1(5): 315-8, 1990).

The unexpected utility of the compositions of the present invention in diminishing or preventing mild cognitive impairment and for preventing further deterioration in subjects already suffering from mild cognitive impairment was hitherto unappreciated.

The archetypical compound dexanabinol was tested in individuals suffering from severe traumatic brain injury (Knoller N. et al., Crit. Care Med. 30(3): 548-54, 2002). As a result of those studies it was discerned that a trend existed for the dexanabinol treated patients to achieve better neurological outcome as assessed by the Galveston Orientation and Amnesia Test, though this was not significant statistically.

It was therefore conceived by the present inventors that these non-psychotropic cannabinoid analogs, having combined neuroprotective and anti-inflammatory attributes might serve to prevent cognitive impairment resulting from secondary or indirect brain injury rather than frank brain injury. According to the present invention, it was undertaken to establish whether pretreatment with these non-psychotropic cannabinoids could decrease the prevalence of mild cognitive impairment resulting from medical procedures outside the central nervous system (CNS). It is now disclosed for the first time that the compounds have remarkable beneficial affects in prevention of cognitive impairment resulting from secondary insults to the brain, rather than direct injury to the CNS.

It is further disclosed that the compositions according to the present invention may be used in methods for preventing the deterioration of mild cognitive impairment into frank cognitive impairment and chronic neurodegeneration.

Compounds of the invention and their previously recognized therapeutic activities, including neuroprotective and/or anti-inflammatory properties, were disclosed in U.S. Pat. Nos. 4,876,276, 5,284,867, 5,521,215, 5,538,993, 5,635,530, 5,932,610, 6,096,740, 6,331,560, 6,545,041 and 6,610,737. Currently preferred compounds were disclosed in U.S. Pat. Nos. 4,876,276 and 6,610,737.

In the present specification the term “prodrug” represents compounds which are rapidly transformed in vivo to the parent compounds of formula (I), for example by hydrolysis in blood. Some of the compounds of formula (I) are capable of further forming pharmaceutically acceptable salts and esters. “Pharmaceutically acceptable salts and esters” means any salt and ester that is pharmaceutically acceptable and has the desired pharmacological properties. Such salts include salts that may be derived from an inorganic or organic acid, or an inorganic or organic base, including amino acids, which is not toxic or undesirable in any way. The present invention also includes within its scope solvates of compounds of formula (I) and salts thereof, for example, hydrates. All of these pharmaceutical forms are intended to be included within the scope of the present invention.

In the present specification “prophylactically effective” is intended to qualify the amount of compound which will achieve the goal of prevention, reduction or eradication of the risk of occurrence of the disorder, while avoiding adverse side effects. The term “therapeutically effective” is intended to qualify the amount of compound that will achieve, with no adverse effects, alleviation, diminished progression or treatment of the disorder, once the disorder can be no further delayed and the patients are no longer asymptomatic. The compositions of the present invention are prophylactic as well as therapeutic.

The “individual” or “patient” for purposes of treatment includes any human or mammalian subject affected by any of the diseases where the treatment has beneficial therapeutic impact.

The most frequent risk factors to develop a given disorder include genetic predisposition, family history of related disorders, predisposing environmental factor, such as a surgical intervention, and age. Epidemiologic studies are performed to refine the characterization of the risk factors for each type of disorder and to define population and individuals at higher risk, for whom prophylactic treatment would clearly be beneficial. The method of the present invention is preferred for use in connection with individuals at substantial or increased risk, relative to the general population, of developing cognitive impairment or neurodegenerative disorders.

The compounds serving as active ingredient in the compositions of the present invention may operate via diverse mechanisms to provide the neuroprotective and anti-inflammatory properties.

By virtue of their combined neuroprotective and anti-inflammatory properties, it is now disclosed that the pharmaceutical compositions of the present invention will be useful in the prevention of post-operative cognitive impairment that may occur in procedures including but not limited to cardiac surgery, valvular surgery, endarterectomy, endovascular therapy, aneurysm repair, orthopedic and prosthetic surgery.

By virtue of their aforesaid therapeutic properties, it is now disclosed that the pharmaceutical compositions of the present invention will be useful in the prevention of disease induced cognitive impairment that may occur in disorders including but not limited to cardiovascular diseases, haemodynamic shock, atherosclerosis, ischemic events, traumatic brain injury (TBI), demyelinating disorders, Huntington's disease (HD), Amyotrophic Lateral Sclerosis (ALS), cancer, autoimmune diseases, multiple sclerosis (MS), mood disorders, epilepsy, chronic infection and viral infections by HCV, HIV, HSV, or CMV.

It is now disclosed that the pharmaceutical compositions of the present invention will be useful in the prevention of therapy induced cognitive impairment that may occur in procedures including but not limited to electroconvulsive therapy and irradiation and during or following the administration of certain medications including but not limited to anesthetic agents, the cytokines used in immunotherapy, the drugs used in chemotherapy, the anti-cholinergic medications, narcotics, opioids, tricyclic antidepressants, anticonvulsants, anti-epileptic drugs such as carbamazepine, valproate, phenytoin, Phenobarbital and gabapentin, histamine H2 receptor antagonists, cardiac medications such as digoxin and beta-blockers, corticosteroids, non steroidal anti-inflammatory drugs (NSAIDs), anti-viral agents such as cyclovir and AZT, anti-parasitic agents and antibiotics.

It is now disclosed that the pharmaceutical compositions of the present invention will be useful in the prevention of neonatal cognitive impairment that may occur in children born pre-term or low weight or having undergone fetal distress.

Since patients suffering from cognitive impairment are at greater risk than the general population to develop neurological disorders such as Alzheimer disease or dementia, the pharmaceutical compositions of the present invention will be useful in prophylactic treatment of populations at risk for neurodegenerative disorders. The risk might be evaluated not only by the occurrence of MCI but also by the presence of other markers for predisposition.

Throughout this specification the alkyl substituents can be saturated or unsaturated, linear branched or cyclic, the latter only when the number of carbon atoms in the alkyl chain is equal or superior to 3.

The pharmaceutical compositions of the present invention, for preventing, alleviating or diminishing post-operative, disease induced, virally induced, therapy induced, neonatal cognitive impairment, comprise as an active ingredient a compound of general formula (I): 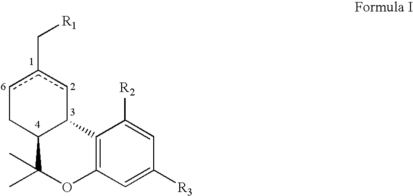
having the (3S,4S) configuration and being essentially free of the (3R,4R) enantiomer, wherein the dashed line indicates an optional C₁-C2 or C6-C1 double bond, and wherein:
R₁ is selected from the group consisting of
   a) R′ where R′ is selected from the group consisting of
      A) a linear or branched, saturated or unsaturated, carbon side chain comprising 1-8 carbon atoms optionally interrupted by 1-3 heteroatoms, and
      B) a saturated or unsaturated cyclic moiety, an aromatic moiety or a heterocyclic moiety; the cyclic moiety having from 3-20 atoms comprising one or two-ringed structures, wherein each ring comprises 3-8 carbons, optionally interrupted by 1-4 heteroatoms, and optionally further substituted with one or more groups selected from
         i) a linear, branched or cyclic, saturated or unsaturated C₁-C₆ alky,
         ii) a linear, branched or cyclic, saturated or unsaturated C₁-C₆ alkoxy,
         iii) a linear, branched or cyclic, saturated or unsaturated C₁-C₆ alkylthio,
         iv) a halogen,
         v) carboxyl,
         vi) —CO₂—C₁-C₆ alkyl, wherein the alkyl can be linear, branched or cyclic, saturated or unsaturated,
         vii) keto,
         viii) nitro,
         ix) a saturated or unsaturated cyclic moiety, or an aromatic or a heterocyclic moiety comprising one or two ringed structures, wherein each ring comprises 3-8 carbons optionally interrupted by 1-4 heteroatoms and is optionally further substituted with one or more groups selected from i)-viii) as defined above,
   b) an amine or an amide substituted with at least one substituent as defined in R′ above,
   c) a thiol, a sulfide, a sulfoxide, a sulfone, a thioester or a thioamide optionally substituted with one substituent as defined in R′ above, and
   d) a hydroxyl or an ether —OR′ wherein R′ is as defined above;
R₂ is selected from the group consisting of
   a) a halogen,
   b) a linear, branched or cyclic, saturated or unsaturated C₁-C₆ alkyl, and
   c) —OR wherein R is selected from the group consisting of
      A) —R″, wherein R″ is hydrogen or a linear, branched or cyclic, saturated or unsaturated C₁-C₆ alkyl optionally containing a terminal —OR′″ or —OC(O)R′″ moiety wherein R′″ is hydrogen or a linear, branched or cyclic, saturated or unsaturated C₁-C₆ alkyl, and
      B) —C(O)R′″ wherein R′″ is as previously defined; and
R₃ is selected from the group consisting of
   a) a linear, branched or cyclic, saturated or unsaturated C₁-C₁₂ alkyl,
   b) —OR^{a}, in which R^{a} is a linear, branched or cyclic, saturated or unsaturated C₂-C₉ alkyl which may be substituted at the terminal carbon atom by a phenyl group, and
   c) a linear, branched or cyclic, saturated or unsaturated C₁-C₇ alkyl-OR′″ wherein R′″ is as previously defined;
and pharmaceutically acceptable salts, esters or solvates thereof.

According to a currently preferred embodiment, we now disclose a pharmaceutical composition for preventing, alleviating or diminishing post-operative, disease induced, virally induced, therapy induced, neonatal cognitive impairment, comprising as an active ingredient a compound of the general formula I wherein R₁ is OH, R₂ is OH, R₃ is 1,1-dimethylheptyl and there is a double bond between C6 and C1. The compound of said pharmaceutical composition is designated hereinafter as HU-211, also known as dexanabinol.

According to another currently preferred embodiment, we now disclose a pharmaceutical composition for preventing, alleviating or diminishing post-operative, disease induced, virally induced, therapy induced, neonatal cognitive impairment, comprising as an active ingredient a compound of the general formula I wherein R₁ is 2-sulfanyl-1H-imidazole, R₂ is OH, R₃ is 1,1-dimethylheptyl and there is a double bond between C6 and C1. The compound of said pharmaceutical composition is designated hereinafter as PRS-211,092.

According to another currently preferred embodiment, we now disclose a pharmaceutical composition for preventing, alleviating or diminishing post-operative, disease induced, virally induced, therapy induced, neonatal cognitive impairment, comprising as an active ingredient a compound of the general formula I wherein R₁ is imidazole, R₂ is OH, R₃ is 1,1-dimethylheptyl and there is a double bond between C6 and C1. The compound of said pharmaceutical composition is designated hereinafter as PRS-211,095.

According to another currently preferred embodiment, we now disclose a pharmaceutical composition for preventing, alleviating or diminishing post-operative, disease induced, virally induced, therapy induced, neonatal cognitive impairment, comprising as an active ingredient a compound of the general formula I wherein R₁ is pyrazole, R₂ is OH, R₃ is 1,1-dimethylheptyl and there is a double bond between C6 and C1. The compound of said pharmaceutical composition is designated hereinafter as PRS-211,220.

The pharmaceutical compositions of the present invention, for preventing, reducing or delaying the deterioration of mild cognitive impairment to chronic neurodegenerative disorders, comprise as an active ingredient a compound of general formula (I): 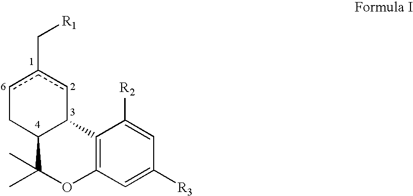
having the (3S,4S) configuration and being essentially free of the (3R,4R) enantiomer, wherein the dashed line indicates an optional C1-C2 or C6-C1 double bond, and wherein:
R₁ is selected from the group consisting of
   a) R′ where R′ is selected from the group consisting of
      A) a linear or branched, saturated or unsaturated, carbon side chain comprising 1-8 carbon atoms optionally interrupted by 1-3 heteroatoms, and
      B) a saturated or unsaturated cyclic moiety, an aromatic moiety or a heterocyclic moiety; the cyclic moiety having from 3-20 atoms comprising one or two-ringed structures, wherein each ring comprises 3-8 carbons, optionally interrupted by 1-4 heteroatoms, and optionally further substituted with one or more groups selected from
         i) a linear, branched or cyclic, saturated or unsaturated C₁-C₆ alkyl,
         ii) a linear, branched or cyclic, saturated or unsaturated C₁-C₆ alkoxy,
         iii) a linear, branched or cyclic, saturated or unsaturated C₁-C₆ alkylthio,
         iv) a halogen,
         v) carboxyl,
         vi) —CO₂—C₁-C₆ alkyl, wherein the alkyl can be linear, branched or cyclic, saturated or unsaturated,
         vii) keto,
         viii) nitro,
         ix) a saturated or unsaturated cyclic moiety, or an aromatic or a heterocyclic moiety comprising one or two ringed structures, wherein each ring comprises 3-8 carbons optionally interrupted by 1-4 heteroatoms and is optionally further substituted with one or more groups selected from i)-viii) as defined above,
   b) an amine or an amide substituted with at least one substituent as defined in R′ above,
   c) a thiol, a sulfide, a sulfoxide, a sulfone, a thioester or a thioamide optionally substituted with one substituent as defined in R′ above, and
   d) a hydroxyl or an ether —OR′ wherein R′ is as defined above;
R₂ is selected from the group consisting of
   a) a halogen,
   b) a linear, branched or cyclic, saturated or unsaturated C₁-C₆ alkyl, and
   c) —OR wherein R is selected from the group consisting of
      A) —R″, wherein R″ is hydrogen or a linear, branched or cyclic, saturated or unsaturated C₁-C₆ alkyl optionally containing a terminal —OR′″ or —OC(O)R′″ moiety wherein R′″ is hydrogen or a linear, branched or cyclic, saturated or unsaturated C₁-C₆ alkyl, and
      B) —C(O)R′″ wherein R′″ is as previously defined; and
R₃ is selected from the group consisting of
   a) a linear, branched or cyclic, saturated or unsaturated C₁-C₁₂ alkyl,
   b) —OR^{a}, in which R^{a} is a linear, branched or cyclic, saturated or unsaturated C₂-C₉ alkyl which may be substituted at the terminal carbon atom by a phenyl group, and
   c) a linear, branched or cyclic, saturated or unsaturated C₁-C₇ alkyl-OR′″ wherein R′″ is as previously defined;
and pharmaceutically acceptable salts, esters or solvates thereof.

According to a currently preferred embodiment, we now disclose a pharmaceutical composition for preventing, reducing or delaying the deterioration of mild cognitive impairment to chronic neurodegenerative disorders, comprising as an active ingredient a compound of the general formula I wherein R₁ is OH, R₂ is OH, R₃ is 1,1-dimethylheptyl and there is a double bond between C6 and C1. The compound of said pharmaceutical composition is designated hereinafter as HU-211, also known as dexanabinol.

According to another currently preferred embodiment, we now disclose a pharmaceutical composition for preventing, reducing or delaying the deterioration of mild cognitive impairment to chronic neurodegenerative disorders, comprising as an active ingredient a compound of the general formula I wherein R₁ is 2-sulfanyl-1H-imidazole, R₂ is OH, R₃ is 1,1-dimethylheptyl and there is a double bond between C6 and C1. The compound of said pharmaceutical composition is designated hereinafter as PRS-211,092.

According to another currently preferred embodiment, we now disclose a pharmaceutical composition for preventing, reducing or delaying the deterioration of mild cognitive impairment to chronic neurodegenerative disorders, comprising as an active ingredient a compound of the general formula I wherein R₁ is imidazole, R₂ is OH, R₃ is 1,1-dimethylheptyl and there is a double bond between C6 and C1. The compound of said pharmaceutical composition is designated hereinafter as PRS-211,095.

According to another currently preferred embodiment, we now disclose a pharmaceutical composition for preventing, reducing or delaying the deterioration of mild cognitive impairment to chronic neurodegenerative disorders, comprising as an active ingredient a compound of the general formula I wherein R₁ is pyrazole, R₂ is OH, R₃ is 1,1-dimethylheptyl and there is a double bond between C6 and C1. The compound of said pharmaceutical composition is designated hereinafter as PRS-211,220.

Specific pharmaceutical compositions of particular interest comprise as an active ingredient compounds of Formula I previously disclosed as HU-211, also known as dexanabinol, U.S. Pat. No. 4,876,276, and PRS-211,092, PRS-211,095, PRS-211,220 in U.S. Pat. No. 6,610,737.

The pharmaceutical compositions contain in addition to the active ingredient conventional pharmaceutically acceptable carriers, diluents and excipients necessary to produce a physiologically acceptable and stable formulation. Some compounds of the resent invention are characteristically hydrophobic and practically insoluble in water with high lipophilicity, as expressed by their high octanol/water partition coefficient expressed as log P values, and formulation strategies to prepare acceptable dosage forms will be applied. Enabling therapeutically effective and convenient administration of the compounds of the present invention is an integral part of this invention.

For water soluble compounds standard formulations will be utilized. Solid compositions for oral administration such as tablets, pills, capsules, softgels or the like may be prepared by mixing the active ingredient with conventional, pharmaceutically acceptable ingredients such as corn starch, lactose, sucrose, mannitol, sorbitol, talc, polyvinylpyrrolidone, polyethyleneglycol, cyclodextrins, dextrans, glycerol, poly-glycolized glycerides, tocopheryl polyethyleneglycol succinate, sodium lauryl sulfate, polyethoxylated castor oils, non-ionic surfactants, stearic acid, magnesium stearate, dicalcium phosphate and gums as pharmaceutically acceptable diluents. The tablets or pills can be coated or otherwise compounded with pharmaceutically acceptable materials known in the art, such as microcrystalline cellulose and cellulose derivatives such as hydroxypropylmethylcellulose (HPMC), to provide a dosage form affording prolonged action or sustained release. Other solid compositions can be prepared as suppositories, for rectal administration. Liquid forms may be prepared for oral administration or for injection, the term including but not limited to subcutaneous, transdermal, intravenous, intrathecal, and other parenteral routes of administration. The liquid compositions include aqueous solutions, with or without organic cosolvents, aqueous or oil suspensions including but not limited to cyclodextrins as suspending agent, flavored emulsions with edible oils, triglycerides and phospholipids, as well as elixirs and similar pharmaceutical vehicles. In addition, the compositions of the present invention may be formed as aerosols, for intranasal and like administration. Topical pharmaceutical compositions of the present invention may be formulated as an aqueous solution, lotion, gel, cream, ointment, emulsion or adhesive film with pharmaceutically acceptable excipients including but not limited to propylene glycol, phospholipids, monoglycerides, diglycerides, triglycerides, polysorbates, surfactants, hydrogels, petrolatum or other such excipients as are known in the art.

Prior to their use as medicaments, the pharmaceutical compositions will generally be formulated in unit dosage form. The active dose for humans is generally in the range of from 0.05 mg to about 50 mg per kg body weight, in a regimen of 1-4 times a day. The preferred range of dosage is from 0.1 mg to about 20 mg per kg body weight. However, it is evident to the man skilled in the art that dosages would be determined by the attending physician, according to the disease to be treated, its severity, the method and frequency of administration, the patient's age, weight, gender and medical condition, contraindications and the like. The dosage will generally be lower if the compounds are administered locally rather than systematically, and for prevention or chronic treatment rather than for acute therapy.

A further aspect of the present invention provides a method of preventing, alleviating or diminishing post-operative, disease induced, virally induced, therapy induced, neonatal cognitive impairment in a patient, by administering a prophylactically and/or therapeutically effective amount of a pharmaceutical composition containing as an active ingredient a compound of general formula (I): 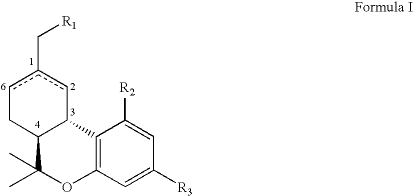
having the (3S,4S) configuration and being essentially free of the (3R,4R) enantiomer, wherein the dashed line indicates an optional C1-C2 or C6-C1 double bond, and wherein:
R₁ is selected from the group consisting of
   a) R′ where R′ is selected from the group consisting of
      A) a linear or branched, saturated or unsaturated, carbon side chain comprising 1-8 carbon atoms optionally interrupted by 1-3 heteroatoms, and
      B) a saturated or unsaturated cyclic moiety, an aromatic moiety or a heterocyclic moiety; the cyclic moiety having from 3-20 atoms comprising one or two-ringed structures, wherein each ring comprises 3-8 carbons, optionally interrupted by 1-4 heteroatoms, and optionally further substituted with one or more groups selected from
         i) a linear, branched or cyclic, saturated or unsaturated C₁-C₆ alkyl,
         ii) a linear, branched or cyclic, saturated or unsaturated C₁-C₆ alkoxy,
         iii) a linear, branched or cyclic, saturated or unsaturated C₁-C₆ alkylthio,
         iv) a halogen,
         v) carboxyl,
         vi) —CO₂—C₁-C₆ alkyl, wherein the alkyl can be linear, branched or cyclic, saturated or unsaturated,
         vii) keto,
         viii) nitro,
         ix) a saturated or unsaturated cyclic moiety, or an aromatic or a heterocyclic moiety comprising one or two ringed structures, wherein each ring comprises 3-8 carbons optionally interrupted by 1-4 heteroatoms and is optionally further substituted with one or more groups selected from i)-viii) as defined above,
   b) an amine or an amide substituted with at least one substituent as defined in R′ above,
   c) a thiol, a sulfide, a sulfoxide, a sulfone, a thioester or a thioamide optionally substituted with one substituent as defined in R′ above, and
   d) a hydroxyl or an ether —OR′ wherein R′ is as defined above;
R₂ is selected from the group consisting of
   a) a halogen,
   b) a linear, branched or cyclic, saturated or unsaturated C₁-C₆ alkyl, and
   c) —OR wherein R is selected from the group consisting of
      A) —R″, wherein R″ is hydrogen or a linear, branched or cyclic, saturated or unsaturated C₁-C₆ alkyl optionally containing a terminal —OR′″ or —OC(O)R′″ moiety wherein R′″ is hydrogen or a linear, branched or cyclic, saturated or unsaturated C₁-C₆ alkyl, and
      B) —C(O)R′″ wherein R′″ is as previously defined; and
R₃ is selected from the group consisting of
   a) a linear, branched or cyclic, saturated or unsaturated C₁-C₁₂ alkyl,
   b) —OR^{a}, in which R^{a} is a linear, branched or cyclic, saturated or unsaturated C₂-C₉ alkyl which may be substituted at the terminal carbon atom by a phenyl group, and
   c) a linear, branched or cyclic, saturated or unsaturated C₁-C₇ alkyl-OR′″ wherein R′″ is as previously defined;
and pharmaceutically acceptable salts, esters or solvates thereof.

According to a currently preferred embodiment, we now disclose a method for preventing, alleviating or diminishing post-operative, disease induced, virally induced, therapy induced, neonatal cognitive impairment in a patient, by administering a prophylactically and/or therapeutically effective amount of a pharmaceutical composition comprising as an active ingredient a compound of the general formula I wherein R₁ is OH, R₂ is OH, R₃ is 1,1-dimethylheptyl and there is a double bond between C6 and C1.

According to another currently preferred embodiment, we now disclose a method for preventing, alleviating or diminishing post-operative, disease induced, virally induced, therapy induced, neonatal cognitive impairment in a patient, by administering a prophylactically and/or therapeutically effective amount of a pharmaceutical composition comprising as an active ingredient a compound of the general formula I wherein R₁ is 2-sulfanyl-1H-imidazole, R₂ is OH, R₃ is 1,1-dimethylheptyl and there is a double bond between C6 and C1.

According to another currently preferred embodiment, we now disclose a method for preventing, alleviating or diminishing post-operative, disease induced, virally induced, therapy induced, neonatal cognitive impairment in a patient, by administering a prophylactically and/or therapeutically effective amount of a pharmaceutical composition comprising as an active ingredient a compound of the general formula I wherein R₁ is imidazole, R₂ is OH, R₃ is 1,1-dimethylheptyl and there is a double bond between C6 and C1.

According to another currently preferred embodiment, we now disclose a method for preventing, alleviating or diminishing post-operative, disease induced, virally induced, therapy induced, neonatal cognitive impairment in a patient, by administering a prophylactically and/or therapeutically effective amount of a pharmaceutical composition comprising as an active ingredient a compound of the general formula I wherein R₁ is pyrazole, R₂ is OH, R₃ is 1,1-dimethylheptyl and there is a double bond between C6 and C1.

A further aspect of the present invention provides a method of preventing, reducing or delaying the deterioration of mild cognitive impairment to chronic neurodegenerative disorders in a patient, by administering a prophylactically and/or therapeutically effective amount of a pharmaceutical composition containing as an active ingredient a compound of general formula (I): 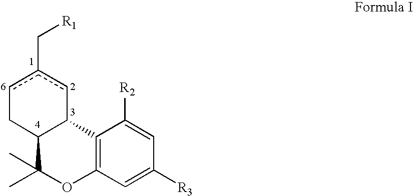
having the (3S,4S) configuration and being essentially free of the (3R,4R) enantiomer, wherein the dashed line indicates an optional C1-C2 or C6-C1 double bond, and wherein:
R₁ is selected from the group consisting of
   a) R′ where R′ is selected from the group consisting of
      A) a linear or branched, saturated or unsaturated, carbon side chain comprising 1-8 carbon atoms optionally interrupted by 1-3 heteroatoms, and
      B) a saturated or unsaturated cyclic moiety, an aromatic moiety or a heterocyclic moiety; the cyclic moiety having from 3-20 atoms comprising one or two-ringed structures, wherein each ring comprises 3-8 carbons, optionally interrupted by 1-4 heteroatoms, and optionally further substituted with one or more groups selected from
         i) a linear, branched or cyclic, saturated or unsaturated C₁-C₆ alkyl,
         ii) a linear, branched or cyclic, saturated or unsaturated C₁-C₆ alkoxy,
         iii) a linear, branched or cyclic, saturated or unsaturated C₁-C₆ alkylthio, ∝iv) a halogen,
         v) carboxyl,
         vi) —CO₂—C₁-C₆ alkyl, wherein the alkyl can be linear, branched or cyclic, saturated or unsaturated,
         vii) keto,
         viii) nitro,
         ix) a saturated or unsaturated cyclic moiety, or an aromatic or a heterocyclic moiety comprising one or two ringed structures, wherein each ring comprises 3-8 carbons optionally interrupted by 1-4 heteroatoms and is optionally further substituted with one or more groups selected from i)-viii) as defined above,
   b) an amine or an amide substituted with at least one substituent as defined in R′ above,
   c) a thiol, a sulfide, a sulfoxide, a sulfone, a thioester or a thioamide optionally substituted with one substituent as defined in R′ above, and
   d) a hydroxyl or an ether —OR′ wherein R′ is as defined above;
R₂ is selected from the group consisting of
   a) a halogen,
   b) a linear, branched or cyclic, saturated or unsaturated C₁-C₆ alkyl, and
   c) —OR wherein R is selected from the group consisting of
      A) —R″, wherein R″ is hydrogen or a linear, branched or cyclic, saturated or unsaturated C₁-C₆ alkyl optionally containing a terminal —OR′″ or —OC(O)R′″ moiety wherein R′″ is hydrogen or a linear, branched or cyclic, saturated or unsaturated C₁-C₆ alkyl, and
      B) —C(O)R′″ wherein R′″ is as previously defined; and
R₃ is selected from the group consisting of
   a) a linear, branched or cyclic, saturated or unsaturated C₁-C₁₂ alkyl,
   b) —OR^{a}, in which R^{a} is a linear, branched or cyclic, saturated or unsaturated C₂-C₉ alkyl which may be substituted at the terminal carbon atom by a phenyl group, and
   c) a linear, branched or cyclic, saturated or unsaturated C₁-C₇ alkyl-OR′″ wherein R′″ is as previously defined;
and pharmaceutically acceptable salts, esters or solvates thereof.

According to a currently preferred embodiment, we now disclose a method for preventing, reducing or delaying the deterioration of mild cognitive impairment to chronic neurodegenerative disorders, by administering a prophylactically and/or therapeutically effective amount of a pharmaceutical composition comprising as an active ingredient a compound of the general formula I wherein R₁ is OH, R₂ is OH, R₃ is 1,1-dimethylheptyl and there is a double bond between C6 and C1.

According to another currently preferred embodiment, we now disclose a method for preventing, reducing or delaying the deterioration of mild cognitive impairment to chronic neurodegenerative disorders, by administering a prophylactically and/or therapeutically effective amount of a pharmaceutical composition comprising as an active ingredient a compound of the general formula I wherein R₁ is 2-sulfanyl-1H-imidazole, R₂ is OH, R₃ is 1,1-dimethylheptyl and there is a double bond between C6 and C1.

According to another currently preferred embodiment, we now disclose a method for preventing, reducing or delaying the deterioration of mild cognitive impairment to chronic neurodegenerative disorders, by administering a prophylactically and/or therapeutically effective amount of a pharmaceutical composition comprising as an active ingredient a compound of the general formula I wherein R₁ is imidazole, R₂ is OH, R₃ is 1,1-dimethylheptyl and there is a double bond between C6 and C1.

According to another currently preferred embodiment, we now disclose a method for preventing, reducing or delaying the deterioration of mild cognitive impairment to chronic neurodegenerative disorders, by administering a prophylactically and/or therapeutically effective amount of a pharmaceutical composition comprising as an active ingredient a compound of the general formula I wherein R₁ is pyrazole, R₂ is OH, R₃ is 1,1-dimethylheptyl and there is a double bond between C6 and C1.

A further aspect of the present invention relates to the use for the manufacture of a medicament for preventing, alleviating or diminishing post-operative, disease induced, virally induced, therapy induced, neonatal cognitive impairment, of a compound of general formula I as previously defined.

A further aspect of the present invention relates to the use for the manufacture of a medicament for preventing, reducing or delaying the deterioration of mild cognitive impairment to chronic neurodegenerative disorders, of a compound of general formula I as previously defined.

The principles of the present invention will be more fully understood in the following examples, which are to be construed in a non-limitative manner.

### EXAMPLES

### Example 1

Neuroprotection by Dexanabinol Following Single High Dose Injection of CREMOPHOR EL®: Ethanol in Rats

The purpose of this study was to characterize the potential of dexanabinol to protect against neuronal vacuolization and cell death in the retrosplenial and cingulated neocortex regions of the brain in Sprague Dawley rats following intravenous administration of a single high dose of CREMOPHOR EL®:ethanol. It has previously been reported that neuronal vacuolization and cell death are among the commonly detected adverse side effects of NMDA antagonists in rodents (Olney J. W. et al., Science 244: 1360-2, 1989). It is now disclosed that the organic cosolvent vehicle used for administration may also induce similar damage in the absence of an NMDA antagonist.

As demonstrated herein the ability of dexanabinol to protect animals against neuronal necrosis induced by CREMOPHOR EL®:ethanol further enhances its potential as a neuroprotective drug. Positive outcome in this model is indicative of the neuroprotective activity of the test compound in human clinical settings. It is now disclosed for the first time that this model of neuronal necrotic damage may serve as a model for focal brain damage and its sequel of cognitive impairment for instance in situations where the patient is in danger of microemboli that may compromise neuronal blood supply during surgery. This study was performed in compliance with good laboratory practice standards.

The Experimental Setup

Sprague Dawley rats (140-250 g body weight, Charles River Laboratory, Mich., USA) were maintained under controlled environmental conditions, with 12:12 hour light dark cycle, and had free access to food and water. The animals were acclimated for at least seven days prior to study day 1. Animals were randomly assigned to the following treatment groups: saline (negative control), CREMOPHOR EL®:ethanol (70:30 w/w respectively supplemented with 0.5% w/v dl-α-tocopherol and 0.01% w/v edetic acid; vehicle) and 50 mg/kg dexanabinol. For all groups the volume dosage was 10 ml/kg and the treatments were administered via bolus intravenous injection into the lateral tail vein of animals. CREMOPHOR EL®:ethanol and dexanabinol 50 mg/ml were diluted 1:10 into saline prior to injection. M-801 at a dose of 5 mg/kg administered subcutaneously at a volume dosage of 5 ml/kg was used as positive control, its vehicle being saline. This dose and route of administration of MK-801 have been shown previously to produce neuronal vacuolization in rats.

Each treatment group consisted of six males and six females, which were at first analyzed separately to assess gender influence. Clinical observations, including observations for mortality or moribundity, were recorded pretest, once daily, and when a change was noted during the twice-daily observations. Body weights were recorded pretest and predose on study day 1. Six animals per sex per group per time point were submitted for necropsy at approximately 4 and 12 hours post-dose, and 1, 3, and 7 days post-dose. At the predetermined time points, the animals were humanely euthanized via intraperitoneal injection of 100 mg/kg sodium pentobarbital to achieve deep anesthesia.

Necropsy

Whole body perfusion were performed using heparinized saline for approximately one minute followed by 10% neutral buffered fornalin for approximately eight to ten minutes on all animals. The calvarium was removed, leaving the brain in the cranial cavity. The heads (with the brain in the cranial cavity) or brains (removed only after 24 hours left in situ in 10% neutral buffered formalin) were placed into 10% neutral buffered formalin until further process toward pathological analysis.

Histopathology

The brains from all animals at all time points were processed and evaluated as follows. The brains from the 4 and 12 hours time point animals were removed and sectioned using a rat brain trimming matrix to yield two coronal sections including the posterior cingulated gyrus (retrosplenial area of the brain). All sections were embedded in glycol methacrylate, sectioned at approximately 2 microns, and stained with hematoxylin and eosin. Brains from the 1, 3 and 7 days post-dose animals were removed and sectioned to yield three coronal sections spanning the brain from the posterior cingulate gyrus and extending rostral to the level of the basal nuclei. These sections were embedded in paraffin and sectioned twice at approximately 5-8 microns, with one section stained with hematoxylin and eosin and the other with Fluoro-Jade B, which selectively stains dying neuronal cells. The slides were microscopically evaluated in a blinded fashion. No statistical analyses were performed.

Dexanabinol Prevents Focal Neuronal Necrosis

First it is important to point out the fact that the present study used a single high dose of dexanabinol, namely 50 mg/kg, that represents to the first approximation the highest tolerated dose in rats and is a significant multiple beyond the highest human dose on a pharmacokinetic equivalent basis. Similarly, the amount of vehicle is significantly above the human clinical dose.

No adverse clinical observations were noted for animals in the saline control group. As expected, animals treated with MK-801 exhibited clinical signs post-dose on study day 1 that included ataxia, tremors and prostration. Animals treated with vehicle or dexanabinol exhibited an undistinguishable extent of prostration and decreased activity post-dose on study day 1. These effects were transient and probably related to the high dose of CREMOPHOR EL®:ethanol. All animals survived until their scheduled sacrifice.

There were no gross lesions determined to be related to the test article, positive control or vehicle control. Upon microscopic evaluation, there was no evidence of neuronal vacuolation in the cingulated gyrus or any other microscopic lesion in animals treated with saline. The classical effects of MK-801 in the pyramidal neurons of cortical layers three and four in the cingulated gyrus were confirmed (vacuolation at four to twelve hours post-dose, followed by neuronal necrosis one to seven days post-dose), thereby validating the study design and conduct. The high dose of CREMOPHOR EL®:ethanol used in this study, was found to induce focal necrosis in various portions of the cortex of the brain. These focal areas of necrosis contained necrotic neurons surrounded by a variably vacuolated neuropil. Though the mechanism underlying the pathogenesis of these focal areas of necrosis was not determined, the histopathologist who analyzed the slides in a masked fashion noted that the outcome of the vehicle treated group suggested an embolic pathogenesis. Thus, this study originally aimed at proving the safety of dexanabinol as compared to the known NMDA antagonist MK-801 (Olney J. W. et al., ibid) provided an unexpected model for microembolization of rat brain. As previously stated microemboli are suspected to be the cause of cognitive impairment in patients undergoing certain types of surgery.

Surprisingly, in the dexanabinol treated group the necrotic foci were much less prevalent, though similar in character to those noted in the vehicle control group. The histopathology results for both males and females for all sections are summarized in table 1.

TABLE 1
Average number of vacuoles and necrotic foci per treatment
group at predetermined time points post-dosing.

| | Average number | | Average number | | |
|---|---|---|---|---|---|
| | of vacuoles | | of necrotic foci | | |
| Treatment | 4 hrs | 12 hrs | Day 1 | Day 3 | Day 7 |
| Saline | None | None | None | None | 1 |
| MK-801 | >21 | >17 | 5 | >18 | >16 |
| Vehicle | None | None | >27.5 | >22 | >7 |
| Dexanabinol | None | None | 0.5 | 0.6 | >3 |

As shown in table 1, dexanabinol is much safer than the NMDA antagonist MK-801 with no formation of vacuoles during the first 12 hours post-dosing, at an absolute dose 10 times higher than MK-801. These findings show that not only is dexanabinol devoid of adverse side effects even at doses well above those intended to produce a neuroprotective effect, it is unexpectedly effective at eradicating the damage induced by the vehicle control.

When considering the present study as a model of microembolization caused by a high amount (650 mg/kg) of CREMOPHOR EL®, it was shown that 50 mg/kg dexanabinol has a dramatic and previously unknown type of neuroprotective effect as measured by the significant reduction in the number of necrotic foci, especially during at least the first three days post-dosing. The residual necrosis observed in the dexanabinol treated group was therefore attributable to the vehicle and not to dexanabinol.

The foci observed in vehicle or dexanabinol treated animals differed from the foci observed in the MK-801 or saline treated controls. The results expressed as number of animals showing necrotic foci in the cingulated gyrus are presented in table 2. The results obtained with male and female animals were combined for the preparation of this table.

TABLE 2
Number of animals with necrotic foci per treatment group at
predetermined time points post-dosing.

| Treatment | Day 1 | Day 3 | Day 7 |
|---|---|---|---|
| Saline | 0/12 | 0/12 | 0/12 |
| MK-801 | 8/12 | 11/12 | 8/12 |
| Vehicle | 2/12 | 1/12 | 0/12 |
| Dexanabinol | 0/12 | 0/12 | 0/12 |

As shown in table 2, necrosis of cingulated gyrus neurons, as noted in the vast majority of MK-801 treated animals, was not noted in any of the dexanabinol treated animals. Dexanabinol has totally prevented the occurrence of necrotic foci in the cingulated gyrus one of the area of the brain that could be involved hi cognitive functions. In reports of similar studies in the literature, it was shown that rats treated with compounds devoid of neuronal vacuolization side effects in the cingulated cortex performed as normal animals in the Morris water maze model for cognitive assessment, as opposed to animals treated with MK-801.

In patients with multi-infarct dementia there was a correlation between the reduction of cerebral blood flow in the cingulated cortex, as assessed by CT scanning, and the lower scores obtained in the Jacobs mini mental scale cognitive test. Similarly temporal and locational disorientation, which correlates with memory impairment, was assessed using the mini mental state examination (MMSE) in patients with Alzheimer's disease and was found to significantly correlate with glucose hypometabolism in the cingulate gyrus. Thus various types of damage or abnormal physiological parameters in the cingulate cortex are associated with cognitive impairment and the ability of dexanabinol to prevent necrotic foci in this area of the brain is expected to have a protective effect against cognitive impairment.

### Example 2

Four Vessel Occlusion (4 VO) in Rats

This model was used to test the ability of dexanabinol or related compounds to act as neuroprotectant when administered prior to surgery. The blood supply to the rat brain arrives via the two vertebral and two common carotid arteries. Transient occlusion of all four vessels results in global ischemia, as would occur during cardiac arrest in humans. Global ischemia results in neurological deficits, including short-term memory deficits, and a selective loss of neurons, including pyramidal cells, in the CA1 field of the hippocampus, similarly to the situation observed in MCI. The ability of compounds to reduce the neurological deficits and increase neuronal survival in this model is considered indicative of their potential in preventing brain damage related to cardiac arrest, and is also predictive for ischemic damages and MCI caused by surgery, for instance cardiac surgery performed or not under cardiopulmonary bypass. The 4-vessel occlusion (4 VO) rat model is relatively easy to produce and shows good reproducibility. Transient ischemia causes irreversible injury to a few, specific populations of highly vulnerable neurons.

The Experimental Setup

Sprague-Dawley male rats (180-400 g body weight, Anilab, Israel) were anesthetized using 4% halothane for induction and 1% halothane for maintenance, in a mixture of 70% N₂O and 30% O₂. Animals were submitted to four-vessel occlusion according to the procedure of Pulsinelli et al. (Pulsinelli W. A. et al., Stroke 10: 267, 1979) in a two-stage operation. On the first day the vertebral arteries were occluded. A midline skin incision was performed above the spinal cord behind the skull occipital bone. Muscles were separated and cut until the C1 vertebra was isolated. The alar foramina were located and the vertebral arteries were coagulated via the alar foramina. Muscles and skin were closed in two layers. On the same day, the common carotid arteries (CCA) were isolated through a central neck midline incision. A loose suture material was positioned around them and the skin was closed.

On the second day, the animals were lightly anesthetized using ether. The skin was opened and CCA were closed for 20 minutes with arterial clips. Animals were deprived of food, with free access to water, overnight between the two stages of the study. Loss of righting reflex was the principal criterion for assuring severe forebrain ischemia in the 4 VO model in the rat. Therefore, animals not showing this sign were not included in the study.

The test compound dexanabinol was dissolved in a minimum volume of absolute ethanol and the solution was added dropwise to hydroxypropyl-β-cyclodextrin (HPCD) powder. The ethanol was allowed to evaporate (1 hour at 45° C.) and water was added to produce a solution of 2 mg/ml dexanabinol in 45% HPCD (weight per volume) that was mixed by stirring, sonicated and filtered. Test compound or vehicle control (45% HPCD) were administered i.v. 15 minutes before the onset of CCA occlusion, at dose volume of 4 ml/kg. Each treatment group was composed of at least 10 animals, randomly assigned.

The study comprises the following control groups: “sham”, animals that undergo occlusion of both vertebral arteries, without common carotid arteries occlusion; “untreated”, animals that undergo occlusion of both vertebral and common carotid arteries, but receive no treatment at all; “vehicle”, animals that undergo occlusion of both vertebral and common carotid arteries and were only treated with vehicle 15 minutes before CCA occlusion. Body (rectal) temperature was maintained between 37° C.-38° C. throughout the procedure.

The Behavioral/Neurological Outcome Assessment

Animals were monitored for their general clinical appearance at predetermined time points: before vertebral occlusion; before and 5 hours after CCA occlusion; then 1, 2, and 3 days after CCA occlusion. Body weight was recorded at baseline and at each follow-up session. The neurological score was attributed following the criteria listed in table 3.

TABLE 3
Neurological score.

| | | Normal | |
|---|---|---|---|
| Test | Normal Score | Total | Deficit |
| Equilibrium on horizontal bar | 1 for each paw | 4 | 0 |
| Grasping reflex | 1 for each paw | 4 | 0 |
| Righting of head when tilted | 1 for each side | 2 | 0 |
| Righting when laid on back | 1 | 1 | 0 |
| Spontaneous motility | 2 | 2 | 1 (walk on |
| | | | digit) or 0 |
| | | | (no walking) |
| Absence of forelimb flexion | 1 | 1 | 0 (present) |
| Absence of thorax twisting | 1 | 1 | 0 (present) |
| Absence of ptosis | 1 | 1 | 0 (present) |

The total neurological score was calculated by adding the results of all tests for each animal and the maximal score obtainable for normal animals is 16 points. Then the average total score was calculated for each treatment group as well as the standard error. The differences between the various treatment groups were statistically analyzed using ANOVA followed by post-hoc Kruskal-Wallis test. A value of p<0.05 was considered to be statistically significant.

It was observed that operated non-treated animals and animals treated with vehicle only displayed a similar drastic drop of about 5 points in neurological score five hours after occlusion and a partial recovery in the following days which stabilized with an average neurological score of about 13 points with no significant trend of further improvement. The vehicle treated group still displayed 35% reduction in neurological score as compared to sham operated animals 72 hours post-occlusion. Sham operated animals stably displayed close to maximal total neurological score of 16 points along the study. Animals treated with 8 mg/kg dexanabinol (HU-211) displayed a lower decrease of about 4 points in neurological score 5 hours after occlusion and a full recovery to sham animals levels within 72 hours. At 48 hours post-occlusion, the dexanabinol treated group was already not statistically different from the sham operated group. From 24 hours post-occlusion on, the dexanabinol treated animals have significantly better neurological outcome than vehicle treated or operated untreated animals.

The Histopathological Outcome

Two weeks after the CCA occlusion, animals are euthanized with sodium pentobarbitone 100 mg/kg i.p. The animals are perfused through the heart with heparinized 4% formaldehyde solution in PBS (pH 7.4). Brains are then removed, and kept in the same solution before preparation for histological evaluation. Coronal blocks of the hippocampal area are prepared and 7 μm thick paraffin sections are cut and stained with haematoxylin and eosin. Three subfields of the CA1 area of the hippocampus are evaluated: medial, middle and lateral. Normal healthy pyramidal cells are counted along 0.4 mm on each side, and the two sides added for each field. Thus, total live cells per 0.8 mm for each subfield in each animal are recorded.

The Statistical Analysis

The data is first expressed as mean±standard error and normalized whenever relevant. The differences between the various treatment groups are statistically analyzed using ANOVA followed by post-hoc Fisher test. A value of p<0.05 is considered to be statistically significant.

### Example 3

The Cognitive Impairment Assessment Following 4 VO

One week after CCA occlusion, the cognitive impairment was measured using a Morris water maze apparatus. The ability of the animals to perform in this test is predictive of their learning and memory capacity. A circular metal pool 150 cm in diameter and 74 cm in height was filled with water at 25° C. to a height of 54 cm. A platform 10 cm in diameter was placed 2 cm below the water surface. The pool was divided (virtually) into 4 equal surface areas identified by arbitrary north, south, east and west coordinates. The platform was located in the middle of the southwest quadrant 25 cm from the pool rim. The swim path was monitored by video camera connected to a computer (View Point, France). If a rat failed to find the hidden platform within 60 seconds, it was placed on the platform for 30 seconds. Animals that found the hidden platform were allowed to stay on it for 30 seconds.

Animals were tested in nine consecutive trials one week following the 4 VO and the learning capacity was measured as the amount of time spent by the animal swimming before finding the hidden platform. Each trial was performed for 1 minute and there were 5 minute intervals between the trials. One day later, eight days post-occlusion, the animals were tested again in order to assess their memory.

Three groups of at least 8 animals each were tested in this study: 1) sham operated animals; 2) operated animals treated 15 minutes before CCA occlusion with HPCD vehicle; 3) operated animals treated 15 minutes before CCA occlusion with 8 mg/kg dexanabinol, prepared and injected intravenously as previously described in example 2.

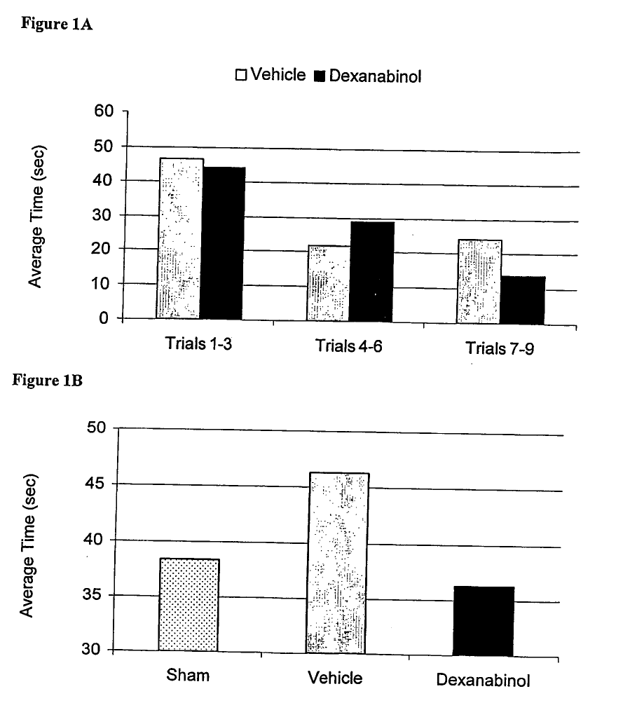

The average latency time in seconds elapsed until platform localization was calculated for each treatment group as an average of three consecutive trials. The results, expressing the learning capacity of the animals, are shown in FIG. 1A. First, the general pattern shows a classical learning curve wherein after each set of trials the animals succeed in performing the task more rapidly, thus validating the study. During the first six trials dexanabinol treated animals learned similarly to vehicle treated animals. However, during the last three trials, dexanabinol treated animals displayed a strong learning advantage over the vehicle control group with more than 42% reduction in average time for platform localization.

The results of the trial performed on day 8 post-occlusion which assessed the memory of the various groups of animals are shown in FIG. 1B. From this figure, it clearly appears that dexanabinol treated animals have not only improved learning capacity, but also better memory over vehicle treated animals with almost 22% reduction in average time for platform localization. Despite the severity of the ischemia caused in the 4 VO model, the animals treated with 8 mg/kg dexanabinol behaved as well as sham operated animals, or even slightly better though this difference is not statistically significant. On average over the ten trials measuring learning and memory, the sham operated animals needed 34.8 seconds to localize the hidden platform while dexanabinol treated animals performed the same task in 4 seconds less with an average of 30.8 seconds. These results strongly support the protective effect of dexanabinol against cognitive impairment when administered prior to surgery.

### Example 4

Microemboli Injection in Rats

The purpose of this study is to reproduce the physiological conditions observed in patients developing CI following surgery. The first step underlying the process resulting in CI is believed to be microemboli entering the cerebral circulation. In this model the animals are embolized by injection of a suspension of small blood clots, to induce behavioral deficits that can be measured quantitatively. Alternatively, the microischemic foci can be obtained by injection of plastic beads of about 100 μm diameter.

The Experimental Setup

Sprague-Dawley male rats (200-300 g body weight, Harlan, Israel) are anesthetized using 4% halothane for induction and 1% halothane for maintenance, in a mixture of 70% N₂O and 30% 0₂. The embolization procedure is according to Lapchak et al. (Lapchak P. A. et al., Stroke 33: 1411, 2002). The animals are anesthetized, the bifurcation of one carotid artery is exposed, and the external carotid is ligated just distal to the bifuircation. A catheter is then inserted anteretrograde into the common carotid and secured with ligatures. The incision around the catheter is closed so that the distal ends are accessible outside the animal's neck. The catheter is filed with heparanized saline and plugged with an injection cap. The animals are allowed to recover from anesthesia for a minimum of 3 hours so that they are awake and behave normally.

To prepare small clots, blood was drawn from a donor rat and allowed to clot at 37° C. The clot is resuspended in PBS solution containing 0.1% bovine serum albumin and is fragmented with a Polytron (setting 6, 3 seconds). The fragments are sized by sequential filtration down to 100 μm² and resuspended in PBS at a predetermined weight of particles per ml that will cause damage in the hippocampal region of the brain. At the time of intra-arterial injection, the clot particles are rapidly injected through the catheter, and the syringe and catheter system are flushed with 5 ml of saline.

The test compounds, including positive controls, are first dissolved in CREMOPHOR EL®:ethanol (70:30 w/w respectively) and further diluted 1:20 in sterile saline to reach the appropriate dose. Test compounds or vehicle control are administered i.v. 15 minutes before embolization, at dose volume of 5 ml/kg. Each treatment group is composed of at least 8 animals, randomly assigned.

The study comprises the following control groups: “sham”, animals that undergo surgical procedure for catheter insertion but receive only saline; “untreated”, animals injected with microemboli that receive no treatment at all; “vehicle”, animals injected with microemboli that are only treated with vehicle 15 minutes before embolization. Body (rectal) temperature is maintained between 37° C.-38° C. throughout the procedure.

The Behavioral/Neurological Outcome Assessment

Animals' spatial learning and memory capacities are assessed in the Morris water maze system as previously described in example 3.

The Histopathological Outcome

The number of live pyramidal cells in the hippocampus area is determined as previously described.

The Statistical Analysis

The data is first expressed as mean±standard error and normalized whenever relevant. The differences between the various treatment groups are statistically analyzed sing ANOVA followed by post-hoc Fisher test. A value of p<0.05 is considered to be statistically significant.

### Example 5

Clinical Trial Protocol

The purpose of this study is to check the effect of one of the preferred compounds on the incidence and severity of post-surgical cognitive impairment. The clinical trial is performed on patients undergoing elective Coronary Artery Bypass Surgery (CABS), where there is a significant risk to develop post-operative CI. This prospective, double blind, placebo controlled, randomized, parallel group Phase IIa trial assesses in 200 patients undergoing CABS, the efficacy, and the safety, of dexanabinol given as a single intravenous fast infusion. An interim analysis is performed following enrolment of the first 100 subjects.

Subjects who meet the inclusion criteria of age 60 and over, undergoing non-emergency CABS, and language proficient, are eligible for the study. The subjects must not suffer at the time of enrolment from neurological disorders, psychiatric diseases, drug abuse, dementia, or being enrolled in another study.

Patients who signed informed consent and who fulfill inclusion criteria are randomized in a 1:1 ratio to receive dexanabinol (150 mg) or placebo (vehicle only). Dexanabinol is supplied in its vehicle, comprising CREMOPHOR EL®:ethanol (70:30 w/w), supplemented with 0.5% w/v dl-α-tocopherol and 0.01% w/v edetic acid. Dexanabinol and placebo are diluted in sterile saline to generate the administered dosage form. The medication is administered during initiation of the first thoracic incision, by fast drip intravenous infusion or peristaltic pump over a period of 15 minutes. Fifteen minutes prior to the administration of the study medication, patients are given intravenously a mixture of H1 and H2 blockers, to prevent possible supersensitivity reaction to the CREMOPHOR EL® component of the vehicle.

The primary efficacy endpoint of the trial is the reduction in the incidence of post-surgical CI at 6 weeks and 3 months following surgery compared to pre-surgery baseline. Patients are tested by a battery of computerized neuropsychological tests assessing the following parameters: power of attention, speed and quality of working memory, quality of episodic secondary memory and continuity of attention. The primary efficacy analysis is a multiple regression analysis of the mean change scores from baseline to 6 weeks and 3 months on the Power of Attention Factor test scores.

Moreover, patients are submitted to a clinical neurological examination leading to a Neurological Assessment Score (NAS) according to NIH guidelines. A series of questionnaires are administered and also considered as secondary end-points including the Hospital Anxiety and Depression Scale (HADS; adapted from Zigmond A. S. et al., Acta Psychiatr. Scand. 67: 361, 1983), the assessment of Quality of Life (QOL; developed by Flanagan (Flanagan J. C., Archives of Physical Medicine and Rehabilitation 63: 56-9, 1982) and adapted by Buckhardt (Burckhardt C. S. et al., Research in Nursing and Health 12, 347-54, 1989), and the Mini Mental State Examination (MMSE; adapted from Folstein M. F. et al., J. Psychiatr. Res. 12: 196-8, 1975). These parameters are used to compare the two treatment groups at the various visits before and after the surgical procedure, for assessing secondary efficacy. The secondary efficacy is analyzed in multiple regression analysis for each of the parameters measured.

Safety is assessed by comparing the rate of adverse events, classified according to body system, severity, consequent dropout if any, and relation to treatment, between the two groups. Comparison of adverse event rates between the two groups is done using Fisher's Exact test. The clinical laboratory tests (Blood Chemistry, Hematology, Urinalysis) as well as the cardiovascular functions (Heart Rate, Mean Arterial Blood Pressure and Electrocardiogram) are taken into account. Mean Change scores are compared between the two groups using a t-test or Wilcoxon-Mann-Whitney test as appropriate.

The trial is in progress in three medical centers in Israel and as of August 2003 forty-seven patients were enrolled.

Although the present invention has been described with respect to various specific embodiments presented thereof for the sake of illustration only, such specifically disclosed embodiments should not be considered limiting. Many other such embodiments will occur to those skilled in the art based upon applicants' disclosure herein, and applicants propose to be bound only by the spirit and scope of their invention as defined in the appended claims.

1. A method for preventing, alleviating or diminishing cognitive impairment, comprising administering to a patient in need thereof a prophylatically and/or therapeutically effective amount of a pharmaceutical composition comprising as an active ingredient a compound of the general formula (I): 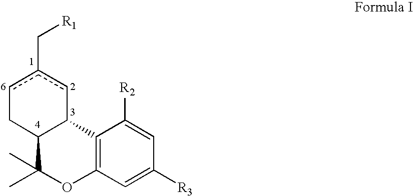
having the (3S,4S) configuration and being essentially free of the (3R,4R) enantiomer, wherein the dashed line indicates an optional C1-C2 or C6-C1 double bond, and wherein:
R₁ is selected from the group consisting of
a) R′ where R′ is selected from the group consisting of
A) a linear or branched, saturated or unsaturated, carbon side chain comprising 1-8 carbon atoms optionally interrupted by 1-3 heteroatoms, and
B) a saturated or unsaturated cyclic moiety, an aromatic moiety or a heterocyclic moiety; the cyclic moiety having from 3-20 atoms comprising one or two-ringed structures, wherein each ring comprises 3-8 carbons, optionally interrupted by 1-4 heteroatoms, and optionally further substituted with one or more groups selected from
i) a linear, branched or cyclic, saturated or unsaturated C₁-C₆ alkyl,
ii) a linear, branched or cyclic, saturated or unsaturated C₁-C₆ alkoxy,
iii) a linear, branched or cyclic, saturated or unsaturated C₁-C₆ alkylthio,
iv) a halogen,
v) carboxyl,
vi) —CO₂—C₁-C₆ alkyl, wherein the alkyl can be linear, branched or cyclic, saturated or unsaturated,
vii) keto,
viii) nitro,
ix) a saturated or unsaturated cyclic moiety, or an aromatic or a heterocyclic moiety comprising one or two ringed structures, wherein each ring comprises 3-8 carbons optionally interrupted by 1-4 heteroatoms and is optionally further substituted with one or more groups selected from i)-viii) as defined above,
b) an amine or an amide substituted with at least one substituent as defined in R′ above,
c) a thiol, a sulfide, a sulfoxide, a sulfone, a thioester or a thioamide optionally substituted with one substituent as defined in R′ above, and
d) a hydroxyl or an ether —OR′ wherein R′ is as defined above;
R₂ is selected from the group consisting of
a) a halogen,
b) a linear, branched or cyclic, saturated or unsaturated C₁-C₆ alkyl, and
c) —OR wherein R is selected from the group consisting of
A) —R″, wherein R″ is hydrogen or a linear, branched or cyclic, saturated or unsaturated C₁-C₆ alkyl optionally containing a terminal —OR′″ or —OC(O)R′″ moiety wherein R′″ is hydrogen or a linear, branched or cyclic, saturated or unsaturated C₁-C₆ alkyl, and
B) —C(O)R′″ wherein R′″ is as previously defined; and
R₃ is selected from the group consisting of
a) a linear, branched or cyclic, saturated or unsaturated C₁-C₁₂ alkyl,
b) —OR^{a}, in which R^{a} is a linear, branched or cyclic, saturated or unsaturated C₂-C₉ alkyl which may be substituted at the terminal carbon atom by a phenyl group, and
c) a linear, branched or cyclic, saturated or unsaturated C₁-C₇ alkyl-OR′″ wherein R′″ is as previously defined;
and pharmaceutically acceptable salts, esters or solvates thereof.

2. The method according to claim 1 wherein the cognitive impairment is post-operative, disease induced, virally induced, therapy induced, or neonatal in origin.

3. The method according to claim 1 wherein R₁ is OH, R₂ is OH, R₃ is 1,1-dimethylheptyl and there is a double bond between C6 and C1.

4. The method according to claim 1 wherein R₁ is 2-sulfanyl-1H-imidazole, R₂ is OH, R₃ is 1,1-dimethylheptyl and there is a double bond between C6 and C1.

5. The method according to claim 1 wherein R₁ is imidazole, R₂ is OH, R₃ is 1,1-dimethylheptyl and there is a double bond between C6 and C1.

6. The method according to claim 1 wherein R₁ is pyrazole, R₂ is OH, R₃ is 1,1-dimethylheptyl and there is a double bond between C6 and C1.

7. The method according to claim 1 wherein the composition is administered orally, parenterally, intravenously, intramuscularly, subcutaneously, transdermally, intrathecally, rectally or intranasally.

8. The method according to claim 1 comprising administering said compound to a patient who undergoes cardiac surgery, valvular surgery, endarterectomy, endovascular therapy, aneurysm repair, orthopedic or prosthetic surgery.

9. The method according to claim 1 comprising administering said compound to a patient who undergoes anesthesia, electroconvulsive-, irradiation-, immuno-, or chemotherapy or to a patient who is treated with anti-cholinergics, narcotics, opioids, tricyclic antidepressants, anticonvulsants, anti-epileptic drugs, histamine H2 receptor antagonists, cardiac medications, digoxin, beta-blockers, corticosteroids, non steroidal anti-inflammatory drugs, anti-viral agents, cyclovir, AZT, anti-parasitic agents or antibiotics.

10. The method according to claim 1 comprising administering said compound to a fetus or neonate who undergoes fetal stress, pre-term-delivery or has low birth weight.

11. A method for preventing, reducing or delaying the deterioration of mild cognitive impairment to chronic neurodegenerative disorders, comprising administering to an individual in need thereof of a prophylatically and/or therapeutically effective amount of a pharmaceutical composition comprising as an active ingredient a compound of the general formula (I): 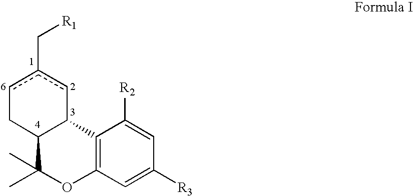
having the (3S,4S) configuration and being essentially free of the (3R,4R) enantiomer, wherein the dashed line indicates an optional C1-C2 or C6-C1 double bond, and wherein:
R₁ is selected from the group consisting of
a) R′ where R′ is selected from the group consisting of
A) a linear or branched, saturated or unsaturated, carbon side chain comprising 1-8 carbon atoms optionally interrupted by 1-3 heteroatoms, and
B) a saturated or unsaturated cyclic moiety, an aromatic moiety or a heterocyclic moiety; the cyclic moiety having from 3-20 atoms comprising one or two-ringed structures, wherein each ring comprises 3-8 carbons, optionally interrupted by 1-4 heteroatoms, and optionally further substituted with one or more groups selected from
i) a linear, branched or cyclic, saturated or unsaturated C₁-C₆ alkyl,
ii) a linear, branched or cyclic, saturated or unsaturated C₁-C₆ alkoxy,
iii) a linear, branched or cyclic, saturated or unsaturated C₁-C₆ alkylthio,
iv) a halogen,
v) carboxyl,
vi) —CO₂—C₁-C₆ alkyl, wherein the alkyl can be linear, branched or cyclic, saturated or unsaturated,
vii) keto,
viii) nitro,
ix) a saturated or unsaturated cyclic moiety, or an aromatic or a heterocyclic moiety comprising one or two ringed structures, wherein each ring comprises 3-8 carbons optionally interrupted by 1-4 heteroatoms and is optionally further substituted with one or more groups selected from i)-viii) as defined above,
b) an amine or an amide substituted with at least one substituent as defined in R′ above,
c) a thiol, a sulfide, a sulfoxide, a sulfone, a thioester or a thioamide optionally substituted with one substituent as defined in R′ above, and
d) a hydroxyl or an ether —OR′ wherein R′ is as defined above;
R₂ is selected from the group consisting of
a) a halogen,
b) a linear, branched or cyclic, saturated or unsaturated C₁-C₆ alkyl, and
c) —OR wherein R is selected from the group consisting of
A) —R″, wherein R″ is hydrogen or a linear, branched or cyclic, saturated or unsaturated C₁-C₆ alkyl optionally containing a terminal —OR′″ —OR(O)R′″ moiety wherein R′″ is hydrogen or a linear, branched or cyclic, saturated or unsaturated C₁-C₆ alkyl, and
B) —C(O)R′″ wherein R′″ is as previously defined; and
R₃ is selected from the group consisting of
a) a linear, branched or cyclic, saturated or unsaturated C₁-C₁₂ alkyl,
b) —OR^{a}, in which R^{a} is a linear, branched or cyclic, saturated or unsaturated C₂-C₉ alkyl which may be substituted at the terminal carbon atom by a phenyl group, and
c) a linear, branched or cyclic, saturated or unsaturated C₁-C₇ alkyl-OR′″ wherein R′″ is as previously defined;
and pharmaceutically acceptable salts, esters or solvates thereof.

12. The method according to claim 11 wherein R₁ is OH, R₂ is OH, R₃ is 1,1-dimethylheptyl and there is a double bond between C6 and C1.

13. The method according to claim 11 wherein R₁ is 2-sulfanyl-1H-imidazole, R₂ is OH, R₃ is 1,1-dimethylheptyl and there is a double bond between C6 and C1.

14. The method according to claim 11 wherein R₁ is imidazole, R₂ is OH, R₃ is 1,1-dimethylheptyl and there is a double bond between C6 and C1.

15. The method according to claim 11 wherein R₁ is pyrazole, R₂ is OH, R₃ is 1,1-dimethylheptyl and there is a double bond between C6 and C1.

16. The method according to claim 11 wherein the composition is administered orally, parenterally, intravenously, intramuscularly, subcutaneously, transdermally, intrathecally, rectally or intranasally.

17. A method for the preparation of a medicament for preventing, alleviating or diminishing cognitive impairment, which comprises associating a compound of the general formula (I) according to claim 1, or pharmaceutically acceptable salts, esters or solvates thereof with a carrier to form a pharmaceutical composition and providing the pharmaceutical composition to a subject in need of treatment for cognitive impairment .

18. The method of claim 17 wherein the subject in need of treatment is experiencing cognitive impairment associated with cardiac surgery valvular surgery, endarterectomy, endovascular therapy, aneurysm repair, orthopedic or prosthetic surgery.

19. The method of claim 17 wherein the subject in need of treatment is experiencing cognitive impairment associated with anesthesia, electroconvulsive-, irradiation-, immuno-, or chemo-therapy, or medication with anti-cholinergics, narcotics, opioids, tricyclic antidepressants, anticonvulsants, anti-epileptic drugs, histamine H2 receptor antagonists, cardiac medications, digoxin, beta-blockers, corticosteroids, non steroidal anti-inflammatory drugs, anti-viral agents, cyclovir, AZT, anti-parasitic agents or antibiotics.

20. The method of claim 17 wherein the subject in need of treatment is experiencing cognitive impairment associated with fetal stress, pre-term-delivery or low birth weight.
